(19) United States
(12) Patent Application Publication
(10) Pub. No.: US 2023/0347039 A1
(43) Pub. Date: Nov. 2, 2023

(54) WOUND-CLEANSING PAD APPARATUS AND METHODS OF USE

(71) Applicant: Farrow Medtech, LLC, College Station, TX (US)

(72) Inventors: Wade P. Farrow, Charlotte, NC (US); Jane Lisak, Hollywood, FL (US)

(21) Appl. No.: 18/140,414

(22) Filed: Apr. 27, 2023

Related U.S. Application Data
(60) Provisional application No. 63/336,661, filed on Apr. 29, 2022.

(51) Int. Cl. A61M 1/00; A61L 15/42; A61F 13/00

(52) U.S. Cl. CPC A61M 1/915; A61L 15/425; A61F 13/00063

(57) ABSTRACT

An improved wound-cleansing pad that is generally comprised of a scrubbing element, a backing element, and a plurality of welds that create a macro texture on the surface of the scrubbing element. The wound-cleansing pad may be used in conjunction with a suitable cleansing gel, or surfactant gel. The wound-cleaning pad may also include a port that allows a negative pressure to be applied to the wound under the pad.

### RELATED APPLICATIONS

This utility patent application claims the benefit of U.S. Provisional Patent Application Ser. No. 63/336,661, filed on Apr. 29, 2022, which is hereby incorporated by reference in its entirety.

### BACKGROUND

### 2. The Field of the Invention

This invention relates to wound care, and more particularly, to novel systems and methods for cleansing, debridement, and/or desloughing wounds.

### 3. Background

Different types of devitalized tissue commonly appear in wound beds. Such tissue may interrupt granulation and delay healing. For example, devitalized tissue may stimulate the overproduction of matrix metalloproteases (MMPs) and thereby slow the healing process.

Additionally, devitalized tissue may provide an environment favorable to bacteria. Bacterial biofilms grow on 60-90% of chronic wounds and about 6% of acute wounds. Bacterial biofilms are low grade infections, cause chronic inflammation, impair wound healing, and increase risk of cellulitis.

Various technologies and/or methods have been developed to improve wound care. However, those technologies and/or methods have certain drawbacks. For example, ultrasonic debridement (e.g., debridement involving oscillations at about 20 kHz or above) is too expensive to be widely adopted, has a large spray pattern, and involves use of equipment that must be autoclaved between uses. Conversely, pulse lavage is often too untidy for use in clinic or emergency room settings.

Accordingly, what is needed are improved systems and methods for cleansing and/or desloughing wounds and removing bacterial biofilms. It would be an advance in the art to provide a cleansing pad specially designed for debridement and/or desloughing of wounds.

### BRIEF SUMMARY OF THE INVENTION

In accordance with the foregoing, certain embodiments of an improved cleansing pad that may be used to clean and debride wounds and may be used in conjunction with other wound cleaning tools, and methods for production and use in accordance with the invention are described.

A cleansing pad may be comprised primarily of a scrubbing element and a backing element. The scrubbing element may be connected to the backing element by one or more welds, or ultrasonic (thermal) welds. The welds may create a surface texture, or macro texture, on the top of the scrubbing element, which top side may be used to scrub and clean a wound.

A wound-cleansing pad may be comprised of a scrubbing element having a top side and a back side, wherein the scrubbing element is comprised of any suitable reticulated foam material. The pad may also be comprised of a backing element having a front side and rear side, wherein the backing element is comprised of a polymeric film that provides a barrier layer, which blocks liquids and similar materials from passing through the back side of the scrubbing element. The pad may also be comprised of a plurality of welds connecting the back side of the scrubbing element to the front side of the backing element, wherein the plurality of welds creates a surface texture on the top side of the scrubbing element.

In another embodiment, a wound-cleansing pad may be comprised of two separate scrubbing elements. Each of the scrubbing elements may be attached to a single backing element. Each of the two scrubbing elements may have similar or different surface textures. Each of the two scrubbing elements may have similar or different porosities. Also, a wound-cleansing pad may be comprised of any reasonable number of scrubbing elements depending on the intended use of the wound-cleansing pad.

In another embodiment, a wound-cleansing pad may be further comprised of a port, or valve. The port may be connected to the backing element in a manner that allows a gas to pass through the port and through the scrubbing element. This embodiment may allow a gas to pass either way through the scrubbing element and port. For example, a sterilizing gas may be passed through the port and the scrubbing element onto a wound under the pad, or a negative pressure may be applied to the wound through the port and the scrubbing element, thereby creating a vacuum over the wound that may help in the desloughing and debridement of the wound, and/or stimulating granulation tissue formation.

A wound-cleansing pad may be used in a variety of ways. A wound-cleansing pad may be used with a gel, or cleansing gel, to help clean and debride a wound. Any suitable cleansing gel may be used. The selection of a cleansing gel may depend on the desired result after the scrubbing of the wound. A wound-cleansing pad with a port may be used in a way that allows a negative pressure to be applied to a wound.

Thus, there are a variety of embodiments of a wound-cleansing pad and a variety of uses for each type of wound-cleansing pad. This allows for greater options in the treatment and care of various types of wounds.

### BRIEF DESCRIPTION OF THE DRAWINGS

The foregoing features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which:

FIG. 1 is a front plan view of one embodiment of a wound-cleansing pad in accordance with the present invention;

FIG. 2 is a rear plan view of the wound-cleansing pad of FIG. 1;

FIG. 3 is a side cross-sectional view of the wound-cleansing pad of FIG. 1;

FIG. 4 is a rear plan view of another embodiment of a wound-cleansing pad in accordance with the present invention;

FIG. 5 is a side cross-sectional view of the wound-cleansing pad of FIG. 4;

FIG. 6 provides a rear plan view of multiple alternative embodiments of wound-cleansing pads in accordance with the present invention;

FIG. 7A is a top view of one embodiment of a pad in accordance with the present invention that may be used as a postoperative wound dressing with negative pressure;

FIG. 7B is a side view of one embodiment of a pad in accordance with the present invention that may be used as a postoperative wound dressing with negative pressure; and

FIG. 7C is a top view of an alternative embodiment of a pad in accordance with the present invention that may be used as a postoperative wound dressing with negative pressure.

### DETAILED DESCRIPTION OF THE PREFERRED EMBODIMENTS

It will be readily understood that the components of the present invention, as generally described and illustrated in the drawings herein, could be arranged and designed in a wide variety of different configurations, or formulations. Thus, the following more detailed description of the embodiments of the system, products and methods of use of the present invention, as presented in the drawings, are not intended to limit the scope of the invention, as claimed, but is merely representative of various embodiments of the invention. The illustrated embodiments of the invention will be best understood by reference to the drawings, wherein like parts are designated by like numerals throughout.

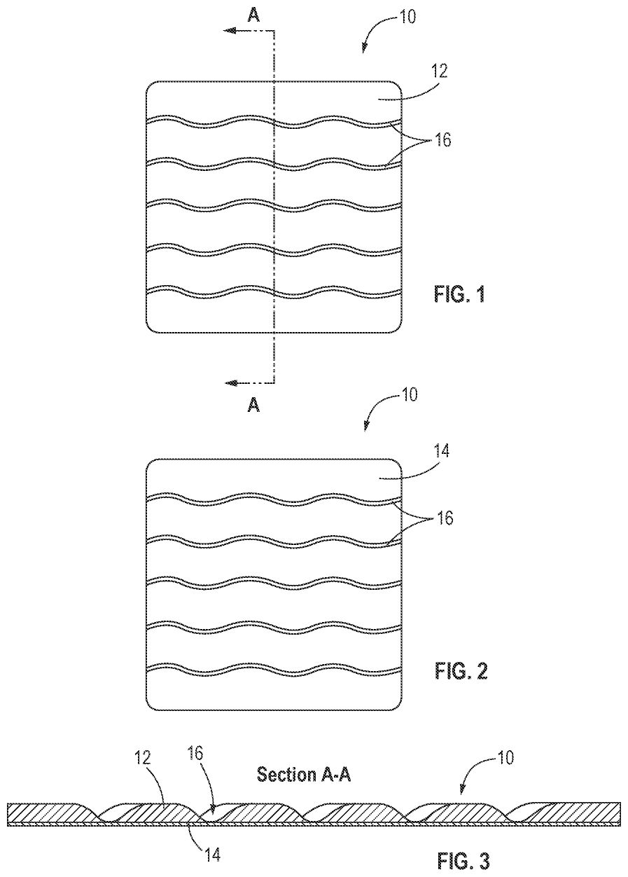

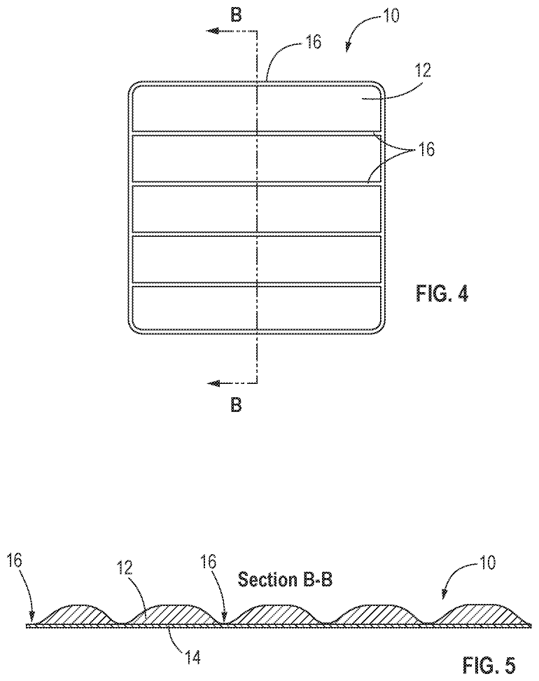

Referring to FIGS. 1-5, devitalized tissue appearing in a wound bed may include fibroslough (primarily fibrinous tissue), leukoslough (primarily WBC accumulation), necroslough (primarily necrotic tissue), and bioslough (primarily biofilm). Slough may be defined as devitalized tissue comprising primarily fibrin, but it may also include white blood cells, debris, and the like. The presence of slough may interrupt granulation and delay healing. It may also provide an environment in which bacteria may grow and spread. There is also an infection continuum in wounds, proceeding from contaminated wound bed (bacteria on surface of wound but not replicating), colonized (bacteria are living on wound bed and replicating), early biofilm formation, mature biofilm formation, and local to systemic infection with biofilm seeding. Accordingly, to promote healing, a wound-cleansing pad 10 in accordance with the present invention may used as a scrubbing tool or part of a scrubbing method to facilitate the removal of devitalized tissue from a wound. This removal may be described as wound cleansing and/or desloughing and/or debridement.

Depending on various factors, devitalized tissue in a wound bed may have different consistencies. For example, the consistency of devitalized tissue may be described as (1) mucinous (i.e., slimy and soft), (2) gelatinous, (3) stringy and/or clumpy, (4) fibrinous, and/or (5) leathery. Depending on the consistency of the devitalized tissue, different methods may be needed. For example, a tool and/or method suitable for debridement of devitalized tissue that is “leathery” may be too aggressive for devitalized tissue that is “mucinous,” or the like. Conversely, a tool and/or method suitable for cleansing and/or desloughing may be too soft to debride devitalized tissue that is fibrinous or leathery. Accordingly, a wound-cleansing pad 10 may be adaptable to provide proper cleansing and/or desloughing of devitalized tissue that is mucinous, gelatinous, stringy, fibrinous, and/or leathery.

Bacterial biofilms can be found in more than about 80% of chronic wounds. Biofilms usually contain multiple bacterial species. Biofilms can be found in multiple places, including within a wound bed (i.e., to a depth of about 60-70 microns below the surface of a wound), on a wound surface, in slough, in fluids on and around a wound bed, etc. and can migrate onto the wound dressings as well. Accordingly, a wound-cleansing pad 10 may be used as a scrubbing and/or abrasive tool to disrupt bacterial growth, prevent the formation of bacterial biofilms, and/or treat bacterial biofilms.

Wound care may be complicated by the fact that a wide variety of people with a wide variety of medical experience and/or training are tasked with providing such care. For example, at home, patients with little to no medical experience or training are often tasked with providing their own wound care (e.g., tasked with fitting and/or changing their own wound dressings). For patients with more serious wounds, wound care may be provided at their respective homes by ancillary healthcare staff having various levels of wound-care training. Some ancillary healthcare staff may have associate nursing degrees. Others have bachelor degrees in nursing. Still others may be specialized wound, ostomy, and continence nurses (WOCN). However, regardless of such educational background, ancillary healthcare staff will have different comfort levels and skill sets with respect to cleansing wounds and conducting even basic wound cleansing and/or debridement.

In institutional, long-term care settings such as nursing homes and long-term acute care (LTAC) hospitals, there may be wound-care teams with more advanced training and an enhanced skill set due to their repeated and regular treating of complicated wounds. In the offices of physicians and surgeons, wounds often need cleansing or mild surgical debridement. Such treatment is often performed in an operating room. However, such use of an operating room is often unnecessary and increases the cost of treatment and may increase morbidity depending on choice of anesthesia.

Accordingly, to address the wide variety of people tasked with providing wound care, a wound-cleansing pad 10 in accordance with the present invention may be easy to use. Moreover, it may be configured so as to limit the potential for misuse or harmful use. Accordingly, both an inexperienced patient and a highly trained medical professional may both use a wound-cleansing pad 10 to beneficial effect.

A wound-cleansing pad 10 in accordance with the present invention may have any suitable shape and size. Suitable shapes may include rectangular, square, rectangular or square with rounded corners, circular, elliptical, triangular, or the like. Suitable sizes may include lengths and widths in a range from about 40 mm to about 100 mm and thickness in a range from about 3 mm to about 10 mm. For example, in certain embodiments, there may be two different sizes of wound-cleansing pads: a large size wound-cleansing pad 10 may be a square with rounded corners that has a width/length of about 80 mm and a thickness of about 5 mm; and a smaller size wound-cleansing pad 10 may be square with rounded corners that has a width/length of about 50 mm and a thickness of about 5 mm.

In selected embodiments, a wound-cleansing pad 10 may include a scrubbing element 12 and a backing element 14. A scrubbing element 12 may be or comprise relatively porous or reticulated foam. For example, a scrubbing element 12 may comprise a relatively thin sheet (e.g., a sheet having a thickness in a range from about 3 mm to about 10 mm) of open cell foam (e.g., reticulated foam formed of polyethylene, polyether, polyurethane, or the like) having a porosity in a range from about 15 pores-per-inch to about 40 pores-per-inch. In one embodiment, a scrubbing element may be approximately 5 mm thick and a porosity of 35 pores per inch, although any dimensions may be utilized. In other embodiments, the scrubbing element may be about 4 mm to about 5 mm thick, and a porosity of about 20 pores-per-inch. Different wound-cleansing pads 10 may include scrubbing elements 12 of different porosity, stiffness, abrasiveness (e.g., via coatings applied to a porous foam structure or thickness of the foam pores/polyurethane type to increase abrasiveness), or the like or a combination or sub-combination thereof. In other embodiments, a scrubbing element 12 may include an anti-microbial agent that may protect the pad 10 from fungal and/or bacterial growth. A would-cleansing pad 10 may be comprised of one or more scrubbing elements 12, where a separate material is used to provide separate and distinct scrubbing elements with a single wound-cleansing pad 10. For example, a wound-cleansing pad 10 may be comprised of a first scrubbing element 12 having a porosity of approximately 15 pores-per-inch and a second scrubbing element 12 having a porosity of approximately 40 pores-per-inch. Accordingly, assembling a wound-cleansing pad 10 with a scrubbing element 12 having particular characteristics may define or control how gently or aggressively the wound-cleansing pad 10 cleanses or desloughs a wound of a patient.

A backing element 14 may be or comprise a barrier layer. For example, a backing element 14 may be or comprise a polymeric film. In selected embodiments, a backing element 14 may be a polyethylene terephthalate (PET) film that is about 0.01 mm (0.5 mil) to about 0.13 mm (5 mil) thick. In one embodiment, a backing element 14 may be a polyethylene terephthalate (PET) film that is about 0.05 mm (2 mil) thick. In one embodiment, a backing element 14 may have a thickness of between about 0.002 inches (0.05 mm) and about 0.01 inches (0.25 mm), and may be comprised of a polyester film. A backing element 14 may be comprised of any suitable material with any suitable dimensions. A backing element 14 may be secured to a scrubbing element 12 in any suitable manner. In selected embodiments, a backing element 14 may be “quilted” using a heat press (i.e., thermal) or ultrasonic weld 16. For example, a thermal press with a suitable configuration may be used to secure the scrubbing element 12 to the backing element 14, wherein the temperature may be about 500° F., the pressure applied may be about 500-1000 lbs., and the pressure may be applied for approximately 20-40 seconds.

A backing element 14 may impart a certain structural strength or a slight resistance to bending that may support a scrubbing element 12 during use of a corresponding wound-cleansing pad 10. Alternatively, or in addition thereto, a backing element 14 may block cleansing liquids, cleansing gels, or the like from passing through a scrubbing element 12 during use of a wound-cleansing pad 10. That is, cleansing liquids, cleansing gels, or the like may aid in cleansing or desloughing a wound. Without a backing element 14, such materials may easily pass through a scrubbing element 12 (e.g., a scrubbing element 12 formed of a relatively thin sheet of porous foam) and leave a working interface between the scrubbing element 12 and the wound without the benefit of those materials. Accordingly, a backing element 14 may prevent or block such unwanted migration of cleansing liquids, cleansing gels, or the like.

In selected embodiments, welds 16 or other connection mechanisms or methods may create texture (e.g., macro texture) for a wound-cleansing pad 10. Such texture may have a larger scale and scope than the porosity (e.g., micro texture) of a scrubbing element 12. For example, welds 16 or other connection mechanisms or methods may create or be associated with indentations or compressions in a scrubbing element 12. A weld 16 may be described as forming a line, a curve, a dot, and/or the like on the surface of a scrubbing element 12. Accordingly, as a wound-cleansing pad 10 is moved back and forth across a wound bed, the overall or macro texture of the pad 10 may facilitate better cleansing (e.g., create high and low pressure areas that facilitate cleansing and/or desloughing).

In certain embodiments, a plurality of wound-cleansing pads 10 may be packaged and/or provided in a kit with a quantity of cleansing liquids, cleansing gels, or the like. In selected embodiments, a dry cleansing agent or surfactant may be applied to a scrubbing element 12 in a manufacturing process. With the addition of a liquid (e.g., water, saline, mineral oil, or the like) to such a scrubbing element 12 at a time of use, the cleansing agent may hydrate and/or dissolve and facilitate or improve a wound-cleansing or wound-desloughing process. In certain embodiments, cleansing liquids, cleansing gels, dry cleansing agents, antiseptic(s) or the like may include a surfactant that may compliment a scrubbing or cleansing action of a wound-cleansing pad 10 and assist in breaking down biofilms. In other embodiments, a wound-cleansing pad 10 may include only the foam, the backing material, at least one surfactant, plus optionally an antiseptic. Suitable surfactants may include nonionic, ionic, or cationic surfactants. Suitable antiseptics may include PHMB, Hypochlorous, methylene blue, silver ions, and iodine. In selected embodiments, an antiseptic may be dried onto (e.g., powdered onto, applied via a solution where the solvent is later evaporated off) a wound-cleansing pad 10.

In certain embodiments, six wound-cleansing pads 10 may be packaged and/or provided in a kit together with surfactant (e.g., surfactant in gel form) in a quantity sufficient to perform six wound cleansings. This may be sufficient wound-cleansing pads 10 and surfactant to clean a wound three times per week for two weeks. Because biofilms can grow back in as little as 24-72 hours, use of wound-cleansing pads 10 and surfactant in accordance with the present invention three times per week for two weeks (or use of two kits to enable one month of cleansing) may improve wound hygiene, allow better and repeated removal of a biofilm, and assist the patient to (over time) prevent the biofilm from returning due to the fact that inflammatory mediators reduce each time the biofilm is removed.

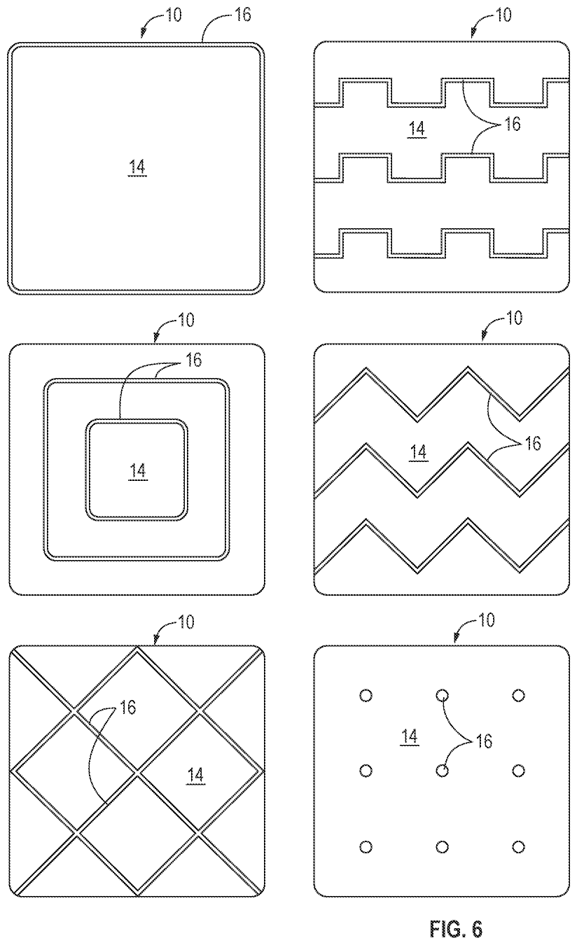

Referring to FIG. 6, welds 16 or other connection mechanisms or methods may have or form any suitable pattern or macro texture, or surface texture, on a wound-cleansing pad 10 in accordance with the present invention. In certain embodiments, welds 16 or other connection mechanisms or methods may simply secure a perimeter of a scrubbing element 12 to a perimeter of a backing element 14. In other embodiments, welds 16 or other connection mechanisms or methods may form lines, line segments, curves, or the like or combinations, sub-combinations, and/or intersections thereof to provide a desired macro texture on a wound-cleansing pad 10. In certain embodiments, a greater number of welds 16 or other connection mechanisms or methods and/or the angles or pointed corners associated therewith may create a more aggressive macro texture. The macro texture, surface texture, may be configured to have a wide variety of designs, including without limitation, parallel lines, parallel waves, concentric rectangles, concentric circles, latticed lines, and the like.

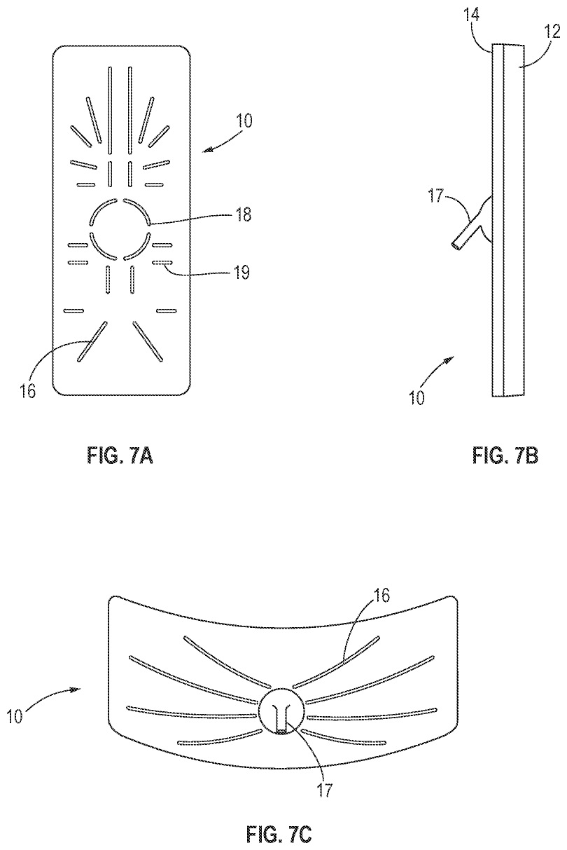

In other embodiments or situations, a device 10 in accordance with the present invention may function as a postoperative wound dressing with negative pressure. FIGS. 7A and 7B illustrate embodiments of such dressings. In these embodiments, the welds 16 may be designed to maximally apply suction in uniform and therapeutic methods across the device 10.

For example, FIG. 7A shows a postoperative dressing for a knee or other joint. The welds 16 are designed to apply wider channels more distally, so that there is even suction applied across the incision. Additionally, other welds 18 are designed to maximize joint movement and patient comfort, for instance having welds designs around the outside of the patella. Other welds 19 are designed to allow better flexion of the device 10.

Referring to FIGS. 7A and 7B, a port 17 can be placed in desirable location, for example more proximally or distally on the device 10. A port 17 may be of any suitable design or configuration and may be composed of any suitable material. Generally, a port 17 may be attached to a backing element 14 in a manner that allows a gas to pass through the scrubbing element 12 and exit the pad 10 through the port 17. This port 17 may provide a connection to negative pressure device directly or through tubing, to apply therapeutic suction to the wound bed, and reduce risk of wound dehiscence postoperatively. Additionally, the dressing 10 may be designed such that suction would draw incision sides together, such as contracting the entire dressing medially towards the middle on both sides, such as to facilitate the device 10 reducing tension on the incision and sutures. Reducing tension on suture lines may be important to reduce risk of wound dehiscence, and pressure necrosis of sutures holding the incision together. Applying a negative pressure may also stimulate granulation tissue formation. The port 17 may be used for any suitable purpose. For example, the port 17 may be utilized to help treat a wound under the pad 10 with a beneficial gas, i.e., a disinfectant, a germicidal, or a sterilizing gas.

FIG. 7C shows another embodiment of a device 10 to function as a postoperative wound dressing for use with negative pressure. In this embodiment, the weld lines 16 are designs for the dressing to conform over an abdomen, such as a low transverse incision for a C-Section incision. These weld lines may also follow natural fold lines in the skin, while also being designed to apply uniform negative pressure to the entire device 10.

In certain embodiments, a surfactant in accordance with the present invention (e.g., a surfactant in gel form) may be a nonionic surfactant. This may be advantageous as nonionic surfactant may be miscible with anionic, cat-ionic, and other nonionic surfactants. Examples of suitable nonionic surfactants may include PLURACARE block copolymers, which are synthetic copolymers of propylene oxide and ethylene oxide. Some suitable grades may include (but are not limited to) Pluragel F 68 (Poloxamer 188), F 108 (Poloxamer 338), F 127 (Poloxamer 407), Poloxamer 124, and Poloxamer 184. Other suitable surfactants may include PEG400, poloxamine 304, poloxamine 904, and poloxamine 908.

Pluronic grade surfactants may have gelation that is thermoreversible. In other words, the surfactant may have a gelatinous consistency when applied to a wound. However, after application and as the surfactant warms with body heat, it may become less gelatinous and more liquid like. This may facilitate application of the surfactant to the patient (e.g., facilitate the surfactant sticking to and not running off a wound surface), while also supporting diffusion of the surfactant across the wound surface and the removal of biofilm and other cellular debris.

In certain embodiments, a surfactant gel may contain polyethylene glycol 400 (polymeric co-surfactant and solvent, which may form stable micelle clusters with Pluronic F127), mineral oil, Pluronic poloxamer F127, phenoxyethanol preservative, and tetrasodium EDTA. The pH may be adjusted for this surfactant gel by use of citric acid, as well as disodium phosphate as a buffering, sequestering agent and pH adjuster base. Because chronic wounds are typically at high pH, even a more neutral range pH of 6-7 may be acceptable, but an acidic gel may counteract the high pH in chronic wounds (e.g., a surfactant gel having a pH of about 4-5 may be preferred in certain embodiments). Overall, a surfactant gel may have a pH in range of about 4 to about 7. Higher acidity may also be achieved by adding an antiseptic such as small amounts of hypochlorite. In such embodiments, the surfactant may better break up and isolate biofilm and bacteria, and the hypochlorite may function as a bactericide. The acidity may also assist in the debridement process by promoting reactions to help break up bonds in the wound bed, resulting in better debridement.

Osmolarity for a surfactant in accordance with the present invention (e.g., a surfactant gel) may be neutral, or hyperosmolar to help collect and absorb drainage from the wound. Normal extracellular fluid has osmolarity of about 280 to about 295 mOsmol/kg. Osmolarity of a surfactant gel may at least about 280 mOsmol/kg, and may be as much as 15% higher than extracellular fluid osmolarity (e.g., as high as 340 mOsmol/kg). Thus, in certain embodiments, a surfactant gel may have an osmolarity range of about 275 to about 340 mOsmol/kg. Nonionic surfactants may be hygroscopic and this may also assist in absorbing wound drainage and thus help trap and remove any inflammatory mediators, bacteria, biofilm, etc. that is broken up and isolated within the micelles.

Referring to Tables 1 and 2, in selected embodiments, a surfactant gel may comprise a combination or sub-combination of trypsin, polyethylene glycol 400, mineral oil, Pluronic Poloxamer F127, phenoxyethanol, tetrasodium EDTA, and water. Trypsin dosage may be variable, depending on the age of the patient. Thus, different formulations may have different amounts of typsin. In certain embodiments, trypsin and/or one or more of the other ingredients may be omitted or have a similar product or ingredient substituted in its place.

TABLE 1

| Ingredient | Weight Percent |
|---|---|
| Trypsin | about 0.005% to about 0.15% |
| Polyethylene Glycol 400 | about 2.5% to about 7.5% |
| Mineral Oil | about 4.4% to about 13% |
| Pluronic Poloxamer F127 | about 10% to about 32% |
| Phenoxyethanol | about 0.5% to about 1.5% |
| Tetrasodium EDTA | about 0.05% to about 0.15% |
| Water | about 32% to about 96% |

TABLE 2

| Ingredient | Weight Percent |
|---|---|
| Trypsin | about 0.01% or omitted |
| Polyethylene Glycol 400 | about 5% |
| Mineral Oil | about 9% |
| Pluronic Poloxamer F127 | about 21% |
| Phenoxyethanol | about 1% |
| Tetrasodium EDTA | about 0.1% |
| Water | about 64% |

In selected embodiments, a surfactant gel may be formed by (1) adding water to a main container, (2) adding tetrasodium EDTA to the main container, (3) mixing until all the solids are dissolved, (4) adding polyethylene glycol 400 to the main container and mixing until uniform, (5) adding trypsin to the main container and mixing until uniform, (6) adding mineral oil to the main container and mixing until uniform, (7) cool the main container to about 5 degrees Celsius, (8) adding the Pluronic Poloxamer F127 and mixing until dissolved, (9) prepare 10% citric acid solution in water and/or a 10% disodium phosphate solution in water, (10) measuring the pH of the gel and adjust the pH to the desired value with either the citric acid or disodium phosphate solutions, (11) keeping the gel refrigerated until packaging, and the like or a sub-combination thereof.

In a surfactant gel in accordance with the present invention, citric acid may be used as a pH adjuster acid. Disodium phosphate may be used as a buffering and sequestering agent and as a pH adjuster base. Polyethylene glycol 400 (e.g., Carbowax) may be a polymeric co-surfactant and solvent due to its ability to form stable micelle clusters with Pluronic F127. Polyethylene glycol 400 may also function at as wound sealing and healing agent, making it a potentially preferred candidate as a co-polymer. Mineral Oil may be used as an emolient, lubricant, therapeutic agent, and/or oleagenous oil that may assist in wound healing and/or play role in attracting bacteria onto itself. Pluronic Poloxamer F127 may thicken, emulsify, and/or gel the matrix. It may also function as a surfactant that prevents creation of bio-film. Phenoxyethanol may be preservative that is safe for use on the skin and wounds. Tetrasodium EDTA may function as an anti-coagulant. Accordingly, if blood coagulation is desired, then tetrasodium EDTA may be removed in certain formulations (e.g., be replaced by a little more mineral oil).

In one embodiment, a wound-cleansing gel may have a formulation as described in Table 3 below.

TABLE 3

| Ingredient | Weight Percent |
|---|---|
| 0.05% Hypochlorous Acid | about 64.2% |
| Saline solution | |
| Polyethylene Glycol 400 | about 5% |
| Mineral Oil | about 8.7% |
| Pluronic Poloxamer F127 | about 21% |
| Phenoxyethanol | about 1% |
| Tetrasodium EDTA | about 0.1% |

A wound-cleansing gel as described in Table 3 may be prepared in accordance with the following process. Add approximately 160.5 grams of distilled water into a 500 mL glass beaker and start mixing at 600 rpm in an overhead prop mixer. Transfer approximately 0.25 grams of Tetrasodium EDTA to the main beaker while mixing and mix until completely dissolved. Add approximately 2.5 grams of phenoxyethanol to the main beaker and mix until completely dissolved. Add approximately 12.5 grams of polyethylene glycol 400 to the main beaker and mix until completely dissolved. Add approximately 21.54 grams of mineral oil to the main beaker and mix until uniform. Transfer the batch into a freezer and cool to 4° C. while stirring on a magnetic stir plate with a steel bar. Monitor the temperature by a digital thermometer with a probe that is place inside the batch. Remove the batch from the freezer and place it into a large container with ice to maintain the cold temperature with mixing using the prop mixer at 1200 rpm. Add approximately 52.5 grams of Poloxamer F127 to the batch slowly, making sure the temperature of the batch stays between 4° C.-5° C., and mix for at least 60 minutes at 1500 rpm. Prepare a 10% citric acid solution with distilled water. Add the citric acid solution to the batch dropwise while mixing at 1500 rpm until reaching the desired pH of 6.0. Package the batch while the temperature is between 4° C.-5° C. This process may be used to prepare approximately 250 grams of the wound-cleansing gel.

In one embodiment, a wound-cleansing gel may have a formulation as described in Table 4 below.

TABLE 4

| Ingredient | Weight Percent |
|---|---|
| 0.05% Hypochlorous Acid | about 64.2% |
| Saline solution | |
| Polyethylene Glycol 400 | about 5% |
| Mineral Oil | about 5.8% |
| Pluronic Poloxamer F127 | about 21% |
| Phenoxyethanol | about 1% |
| Lidocaine HCl | about 3% |

A wound-cleansing gel as described in Table 4 may be prepared in accordance with the following process. Add approximately 160.5 grams of distilled water into a 500 mL glass beaker and start mixing at 600 rpm in an overhead prop mixer. Add approximately 7.5 grams of Lidocaine HCl and mix until completely dissolved. Add approximately 2.5 grams of phenoxyethanol to the main beaker and mix until completely dissolved. Add approximately 12.5 grams of polyethylene glycol 400 to the main beaker and mix until completely dissolved. Add approximately 14.5 grams of mineral oil to the main beaker and mix until uniform. Transfer the batch into a freezer and cool to 4° C. while stirring on a magnetic stir plate with a steel bar. Monitor the temperature by a digital thermometer with a probe that is place inside the batch. Remove the batch from the freezer and place it into a large container with ice to maintain the cold temperature with mixing using the prop mixer at 1200 rpm. Add approximately 52.5 grams of Poloxamer F127 to the batch slowly, making sure the temperature of the batch stays between 4° C.-5° C., and mix for at least 60 minutes at 1500 rpm. Prepare a 10% citric acid solution with distilled water. Add the citric acid solution to the batch dropwise while mixing at 1500 rpm until reaching the desired pH of 6.0. Package the batch while the temperature is between 4° C.-5° C. This process may be used to prepare approximately 250 grams of the wound-cleansing gel.

A wound-cleansing pad in accordance with the invention may be used in a variety of ways. In one embodiment, a method for cleansing a wound may comprise, providing a wound-cleansing pad, wherein the wound-cleansing pad is comprised of a scrubbing element, a backing element, and a plurality of welds, wherein the scrubbing element is a reticulated foam, the backing element is a polymeric film that provides a barrier layer, and the plurality of welds forms a surface texture on the top of the scrubbing element. The method may further comprise providing a gel, wherein the gel may be comprised of a hypochlorous acid saline solution and a surfactant, or the gel may have any suitable formulation. The method may further comprise applying the gel, whichever gel is selected, to the wound-cleansing pad. The method may further comprise cleansing the wound by applying the wound-cleansing pad with the gel to the wound and removing slough from the wound, which may be accomplished by rubbing or scrubbing the wound with the pad in a manner suitable for desloughing the wound, and/or debridement of the wound. Such a method may be utilized to cleanse a wide variety of wounds, including without limitation, wounds that may be described as mucinous, gelatinous, stringy, fibrinous, and leathery, or the like. Moreover, a desired porosity may be selected for the scrubbing element depending on the type of wound being cleansed. For example, a scrubbing element may have a porosity within the approximate range of 15 pores/inch to 40 pores/inch.

In another embodiment, a method for cleansing a wound may comprise, providing a wound-cleansing pad, wherein the wound-cleansing pad is comprised of a scrubbing element, a backing element, a plurality of welds, and a port, wherein the scrubbing element is a reticulated foam, the backing element is a polymeric film that provides a barrier layer, the plurality of welds forms a surface texture on the top of the scrubbing element, and the port is connected to the backing element in a manner that allows a gas to be passed through the port and through the scrubbing element. The method may further comprise positioning the wound-cleansing pad on a wound and applying a negative pressure through the port to the wound. The method may further comprise positioning the wound-cleansing pad on a wound and applying a gas through the port onto the wound.

In another embodiment, a method for cleansing a wound may comprise, providing a wound-cleansing pad, wherein the wound-cleansing pad is comprised of a scrubbing element, a backing element, a plurality of welds, and a port, wherein the scrubbing element is a reticulated foam, the backing element is a polymeric film that provides a barrier layer, the plurality of welds forms a surface texture on the top of the scrubbing element, and the port is connected to the backing element in a manner that allows a gas to be passed through the port and through the scrubbing element. The method may further comprise providing a gel, wherein the gel may be comprised of a hypochlorous acid saline solution and a surfactant, or the gel may have any suitable formulation. The method may further comprise applying the gel, whichever gel is selected, to the wound-cleansing pad. The method may further comprise cleansing the wound by applying the wound-cleansing pad with the gel to the wound and removing slough from the wound, which may be accomplished by rubbing or scrubbing the wound with the pad in a manner suitable for desloughing the wound, and/or debridement of the wound. The method may further comprise positioning the wound-cleansing pad on a wound and applying a negative pressure through the port to the wound.

The subject invention may be more easily comprehended by reference to the specific embodiments recited herein, which are representative of the invention. The described embodiments are to be considered in all respects only as illustrative, and not restrictive. All changes which come within the meaning and range of equivalency of the described embodiments are to be embraced within their scope. However, it must be understood that the specific embodiments are provided only for the purpose of illustration, and that the invention may be practiced in a manner separate from what is specifically illustrated without departing from its scope and spirit.

What is claimed and desired to be secured by United States Letters Patent is:

1. A wound-cleansing pad, comprising:
a first scrubbing element having a first top side and a first back side, wherein the first scrubbing element is comprised of a first reticulated foam material having a first porosity;
a backing element having a front side and a rear side; and
a first plurality of welds connecting the first back side of the first scrubbing element to the front side of the backing element, wherein the first plurality of welds creates a first surface texture on the first top side of the first scrubbing element.

2. The wound-cleansing pad of claim 1, wherein the backing element is comprised of a polymeric film that provides a barrier layer and the barrier layer blocks liquid materials from passing through the first back side of the first scrubbing element.

3. The wound-cleansing pad of claim 1, further comprising:
a second scrubbing element having a second top side and a second back side, wherein the second scrubbing element is comprised of a second reticulated foam material having a second porosity; and
a second plurality of welds connecting the second back side of the second scrubbing element to the front side of the backing element, wherein the second plurality of welds creates a second surface texture on the second top side of the second scrubbing element.

4. The wound-cleansing pad of claim 2, further comprising:
a second scrubbing element having a second top side and a second back side, wherein the second scrubbing element is comprised of a second reticulated foam material having a second porosity; and
a second plurality of welds connecting the second back side of the second scrubbing element to the front side of the backing element, wherein the second plurality of welds creates a second surface texture on the second top side of the second scrubbing element.

5. The wound-cleansing pad of claim 2, further comprising:
a port connected to the backing element in a manner that allows a gas to be passed through the port and the first scrubbing element.

6. The wound-cleansing pad of claim 5, wherein the port is connected to a source of negative pressure.

7. The wound-cleansing pad of claim 2, wherein the first plurality of welds includes at least one weld designed to accommodate bending of the backing element.

8. The wound-cleansing pad of claim 7, further comprising:
a port connected to the backing element and connected to a source of negative pressure in a manner that allows a negative pressure to be applied through the first scrubbing element.

9. A wound-cleansing pad, comprising:
a scrubbing element having a top side and a back side, wherein the scrubbing element is comprised of a reticulated foam material;
a backing element having a front side and a rear side, wherein the backing element is comprised of a polymeric film that provides a barrier layer and the barrier layer blocks liquid materials from passing through the back side of the scrubbing element;
a plurality of welds connecting the back side of the scrubbing element to the front side of the backing element, wherein the plurality of welds creates a surface texture on the top side of the scrubbing element; and
a port connected to the backing element in a manner that allows a gas to be passed through the port and the scrubbing element.

10. The wound-cleansing pad of claim 9, wherein the port is connected to a source of negative pressure.

11. The wound cleansing pad of claim 9, wherein the surface texture is selected from the group consisting of parallel lines, parallel waves, concentric rectangles, concentric circles, and latticed lines.

12. The wound cleansing pad of claim 9, wherein the reticulated foam material has a porosity of approximately 20 pores-per-inch.

13. The wound cleansing pad of claim 11, wherein the reticulated foam material has a porosity of approximately 35 pores-per-inch.

14. The wound cleansing pad of claim 9, wherein the backing element is comprised of a polyester film that is about 0.076 mm thick.

15. A method for cleansing a wound, comprising:
providing a wound-cleansing pad, wherein the wound-cleansing pad is comprised of a scrubbing element, a backing element, and a plurality of welds, wherein the scrubbing element is a reticulated foam, the backing element is a polymeric film that provides a barrier layer, and the plurality of welds forms a surface texture on the top of the scrubbing element;
providing a gel, wherein the gel is comprised of a hypochlorous acid saline solution, and a surfactant;
applying the gel to the wound-cleansing pad; and
cleansing a wound by applying the wound-cleansing pad with the gel to the wound and removing slough from the wound.

16. The method for cleansing a wound of claim 15, wherein wound-cleansing pad further comprises a port that is connected to the backing element in a manner that allows a gas to be passed through the port and through the scrubbing element.

17. The method for cleansing a wound of claim 16, further comprising:
positioning the wound-cleansing pad on the wound; and
applying a negative pressure through the port to the wound.

18. The method of cleansing a wound of claim 16, further comprising:
positioning the wound-cleansing pad on the wound; and
applying a gas through the port onto the wound.

19. The method of cleansing a wound of claim 15, wherein the wound being cleansed is selected from the group consisting of mucinous, gelatinous, stringy, fibrinous, and leathery.

20. The method of cleansing a wound of claim 19, wherein the scrubbing element has a porosity between 15 pores-per-inch and 40 pores-per-inch.
